(19) United States
(12) Patent Application Publication
(10) Pub. No.: US 2026/0115224 A1
(43) Pub. Date: Apr. 30, 2026

(54) METHODS FOR TREATING COCCYGEAL, HIP, THIGH, OR LEG PAIN WITH HYALURONIC ACID

(71) Applicant: David Donaldson Brown, Garden Grove, CA (US)

(72) Inventor: David Donaldson Brown, Garden Grove, CA (US)

(21) Appl. No.: 19/372,995

(22) Filed: Oct. 29, 2025

Related U.S. Application Data
(60) Provisional application No. 63/713,371, filed on Oct. 29, 2024.

(51) Int. Cl. A61K 31/728; A61K 9/00; A61P 29/00

(52) U.S. Cl. CPC A61K 31/728; A61K 9/0019; A61P 29/00

(57) ABSTRACT

The present invention provides methods for treating hip, thigh, and/or leg pain in a subject, comprising administering to the subject, by one or more injections, a composition comprising hyaluronic acid (HA) and a pharmaceutically acceptable carrier. Also provided are methods for treating pain associated with lower back surgery and methods for treating coccygeal pain.

### CROSS-REFERENCE TO RELATED APPLICATIONS

This application claims priority to U.S. Provisional Application No. 63/713,371, filed on Oct. 29, 2024, the contents of which are herein incorporated by reference in their entirety.

### BACKGROUND

Hip and thigh pain are commonly related to the femoro-acetabular joint, yet there are common, but unrecognized causes such as tears of the fascia lata attachment to the iliac crest (Deshmukh 2019). For the origin of such pains, new and effective treatments are needed. Further, coccygeal pain, often referred to as coccydynia, frequently traumatic in origin, is often difficult to treat. Such treatments are based on limited evidence of effectiveness, and newer treatments are needed (Mazzoleni 2025). Finally, “failed back surgery syndrome” where there is persistent or worsening pain after lumbar spine surgery, is difficult to manage and often refractory to treatment, warranting new methods of treatment.

### SUMMARY

In an aspect, the present invention provides methods for treating pain in a subject. The methods comprise administering to the subject, by one or more injections, a composition comprising a therapeutically effective amount of hyaluronic acid (HA) and a pharmaceutically acceptable carrier. The pain may be nociceptive pain, and the methods may further comprise palpating the iliac crest, posterior superior iliac spine, sacrum and/or coccyx and selecting areas of pain/tenderness for injection.

In a first embodiment, the HA injections are used to treat pain in the hip, thigh, and/or leg. The hip, thigh, and/or leg pain may be associated with lower back pain. In a second embodiment, the HA injections are used to treat pain in the coccygeal region. In a third embodiment, the HA injections are used to treat pain associated with a lower back surgery, wherein the pain includes one or more of lower back, buttocks, sacral region, coccygeal hip, thigh, and/or leg pain that persists after surgery.

### BRIEF DESCRIPTION OF THE DRAWINGS

FIG. 1 is a drawing illustrating the locations of the fleshy gluteal muscle attachments of the gluteus medius [100], gluteus maximus [110], gluteus minimus [120], and piriformis to the sacrum [140], coccyx [150], ilium [160], and iliac crest [170], with the “X” marking locations to be palpated for tenderness and optionally injected with hyaluronic acid. The piriformis originates from the anterior surface of the sacrum and is not injected with hyaluronic acid. Four sites for palpation and optional hyaluronic acid injection of the gluteus medius are shown to the left side of a subject, along the inferior margin of the iliac crest. Seven sites for palpation and optional hyaluronic acid injection of the gluteus maximus are shown to the left side of a subject: two along the posterior superior iliac spine [180], three along the lateral edge/margin of the of the sacrum, and two along the coccyx. Four sites for palpation and optional hyaluronic acid injection of the gluteus minimus are shown to the right side of a subject, along the ilium.

FIG. 2 is a drawing illustrating the medial and superior margins of the posterior superior iliac spine [200] and the superior margin of the iliac crest [210], where the thoracolumbar fascia, specifically the interwoven thoracolumbar composite [220] attaches. Sites marked with an “X are palpated for tenderness and optionally injected with hyaluronic acid. Referring to location [200] along the medial and superior margins of the posterior superior iliac spine, two sites for palpation and optional hyaluronic acid injection are marked. Referring to location [210] along the superior margin of the iliac crest, four sites for palpation and optional hyaluronic acid injection are marked.

FIG. 3 is another drawing illustrating the sites of palpation and injection for treating hip, thigh and/or leg pain by injecting the attachments of the gluteus medius [100] to the iliac crest with hyaluronic acid. Sites marked with an “X” are palpated any optionally injected with hyaluronic acid. Six sites for palpation and optional hyaluronic acid injection are shown to the right side of a subject.

### DETAILED DESCRIPTION

Hyaluronic acid (HA) is a large, naturally occurring polymer that is a major constituent of synovial fluid, tendon, ligament and extracellular matrix and is involved in lubricating joint movements. As used herein, “hyaluronic acid” or “HA” encompasses, HA, its conjugate base (hyaluronate), and derivatives thereof. In addition to its lubricating properties, HA exhibits anti-inflammatory properties that are dependent on concentration and molecular weight.¹²

HA is FDA-approved as an injection treatment for osteoarthritis of the knee. In addition, HA has been investigated extensively for its use in other arthritic joints. HA has been used with some degree of success in several tendinous disorders, including supraspinatus tendinosis, lateral epicondylitis, and patellar tendinopathy.¹³ However, many muscles attach to bone without an intervening tendon, such muscles attach over broad skeletal surface areas, and are referred to as fleshy muscle attachments (Benjamin 2002 in text). The fleshy muscle attachments of the gluteal muscles to the sacrum and ilium, where these muscles attach over a broad surface area without an intervening tendon, have hitherto not been generally recognized as sources of acute or chronic pain of the hip, thigh, leg or coccygeal region. Further, the fleshy muscle attachments of the external oblique, internal oblique, and transversus abdominis to the iliac crest may also contribute to hip pain. The fascia lata, which invests the thigh muscles and encases the tensor fascia lata muscle, is another potential source of pain at its attachment site to the iliac crest (Deshmukh 2019). This structure differs from tendon in that it is composed of multiple aponeurotic layers, with each layer having fibers oriented in one direction but different than the other layers, and each layer being separated by a lubricating layer (Fede 2021).

The present inventor previously discovered that lower back, buttocks, and/or sacral pain is effectively treated with local injections of HA into, or immediately adjacent to, soft tissue attachments to the iliac crest, posterior superior iliac spine, and/or sacrum (PCT/US2024/053392, which is incorporated herein by reference in its entirety). As demonstrated in the Examples, the present inventor discovered that injection of HA into the soft tissue attachments to the coccyx, sacrum, ilium, and/or iliac crest, which attachments may comprise the fleshy muscle attachments of the gluteal muscles, internal oblique, external oblique, transversus abdominus muscles, or the fascia lata attachment to iliac crest, can effectively treat certain forms of coccygeal, hip, thigh, and/or or leg pain. Additionally, injection of hyaluronic acid into the soft tissue attachments of the iliac crest, the posterior superior iliac spine, and/or the sacrum was effective in treating patients with one or more of lower back, sacral, buttocks hip, thigh, and/or leg pain that has persisted after lumbar spine surgery. Thus, the present invention provides methods for treating pain comprising administering one or more HA injections.

Lower Back, Buttocks, Sacral, Hip, and or Thigh Pain that Persists after Lumbar Spine Surgery

In an aspect, the present invention provides methods for treating pain in a subject, wherein the subject is experiencing persistent pain in one or more of the lower back, buttocks, sacral, hip, and/or thigh region after a lumbar spine surgery. The pain may have persisted for at least 1 month, 3 months, 6 months, 1 year, 3 years, 5 years, 10 years, 15 years, 20 years, or for a period within a range bounded by any of the foregoing. In some embodiments, the pain severity has worsened following the surgery. A “subject” or “patient” refers to any organism in need of and/or subjected to a treatment, such as a farm animal, domestic animal, or human. In some embodiments, the subject is a human. As used herein, “lower back” refers to the lumbar spine region between the last thoracic vertebra (T12) and the first sacral vertebra (S1) and extending laterally to include the iliac crests, “sacral area” refers to the region at the base of the spine which comprises the sacrum, and “buttocks” refers to the region below the lower back, including all soft tissue and bone extending to the gluteal sulcus on each side.

In an aspect, the methods comprise administering to the subject a composition comprising a therapeutically effective amount of HA. HA is a polymer of D-glucuronic acid and N-acetyl-D-glucosamine that occurs naturally at varied sizes, with molecular weights ranging from 5 to 20,000 kilodaltons (kDa). The present methods may use HA having any suitable molecular weight. In some embodiments, the composition comprises HA having a molecular weight of about 50, 75, 100, 200, 300, 400, 500, 600, 700, 800, 900, 1000, 5000, 10000, 15000, or 20000 kDa, or a molecular weight within a range bounded by any of the forgoing. In some embodiments, the HA has a molecular weight of at least 500 kDa. In some embodiments, the HA has a molecular weight of about 500 kDa to about 730 kDa, or between about 450 kDa and 800 kDa, or between 400 kDa and 1000 kDa. The methods may suitably use linear HA or cross-linked HA. Suitable polymer cross-linking agents are known in the art and include, without limitation, cross-linking proteins/peptides, 1,4-Butanediol diglycidyl ether, and divinyl sulfone.

In some embodiments, the composition comprises a pharmaceutically acceptable salt of HA. As used herein, a “pharmaceutically acceptable salt” refers to a salt that retains the desired biological activity of the parent compound and does not impart any undesired toxicological effects (see e.g., Berge, S. M., et al. (1977). J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like. In some embodiments, the composition comprises a HA sodium salt.

The HA may be present in the composition at any suitable concentration. In some embodiments, the HA is present in the composition at a concentration of about 1 mg/mL, 5 mg/mL, 10 mg/mL, 15 mg/mL, 20 mg/mL, or 25 mg/mL, or at a concentration within a range bounded by any of the foregoing. In some embodiments, the HA is present in the composition at a concentration of about 10 mg/mL. The concentration of HA in the composition may be between 9-11 mg/mL, between 8-12 mg/mL or between 5-15 mg/mL.

In an aspect, the composition further comprises a pharmaceutically acceptable carrier. Pharmaceutically acceptable carriers are known in the art and include, but are not limited to, diluents (e.g., Tris-HCl, acetate, phosphate), preservatives (e.g., Thimerosal, benzyl alcohol, parabens), solubilizing agents (e.g., glycerol, polyethylene glycerol), emulsifiers, liposomes, and nanoparticles. Pharmaceutically acceptable carriers may be aqueous or non-aqueous solutions, suspensions, or emulsions. Examples of nonaqueous solvents are propylene glycol, polyethylene glycol, vegetable oils such as olive oil, and injectable organic esters such as ethyl oleate. Aqueous carriers include isotonic solutions, alcoholic/aqueous solutions, emulsions, or suspensions, including saline and buffered media. The composition may further comprise additives such as albumin or gelatin to prevent absorption to surfaces, detergents (e.g., Tween 20, Tween 80, Pluronic F68, bile acid salts), anti-oxidants (e.g., ascorbic acid, sodium metabisulfite), bulking substances or tonicity modifiers (e.g., lactose, mannitol). Components of the compositions may be covalently attached to polymers (e.g., polyethylene glycol), complexed with metal ions, or incorporated into or onto particulate preparations of polymeric compounds (e.g., polylactic acid, polyglycolic acid, hydrogels, etc.) or onto liposomes, microemulsions, micelles, milamellar or multilamellar vesicles, erythrocyte ghosts, or spheroplasts. The compositions may also be formulated in lipophilic depots (e.g., fatty acids, waxes, oils) for controlled or sustained release.

In some embodiments, the pharmaceutically acceptable carrier comprises sodium chloride, monobasic sodium phosphate, dibasic sodium phosphate, and water. HA is FDA-approved and commercially available as an injection treatment for osteoarthritis of the knee in several brands of differing molecular weights (including, without limitation, Hyalgan™, Synvisc™, Monovisc™, Orthovisc™, Durolane™ and several others), Thus, in some embodiments, the methods comprise administering a commercially available formulation.

As used herein, the term “therapeutically effective amount” refers to an amount of HA sufficient to treat pain in a subject receiving the composition. Methods for assessing pain treatment are known in the art and include, without limitation, assessments based on pain severity (average and/or maximum) and pain duration. In some embodiments, pain severity is assessed using a visual analog numerical rating scale ranging from 0 (no pain) to 10 (worst pain possible). Average and maximum pain severity may be assessed on a daily, weekly, or monthly basis. In some embodiments, the patient's average pain severity and/or maximum pain severity is reduced following administration of the HA composition. In some embodiments, the total number of hours per day during which the patient experiences pain is reduced following administration of the HA composition. In some embodiments, the number of pain-free days per week and/or month is increased following administration of the HA composition.

The HA composition may be administered to a patient experiencing acute pain or chronic pain. As used herein, “chronic pain” refers to pain that has persisted for a duration of at least 3 months, while “acute pain” refers to sudden, temporary pain that typically resolves in less than 3 months. The HA composition may be administered to a patient experiencing pain of any severity. In some embodiments, the HA composition is administered to a patient experiencing one or more of chronic lower back, buttocks, sacral, hip, thigh or leg region pain that persist after lumbar spine surgery, with a daily maximum pain severity of at least 7/10 on the visual analog numerical rating scale.

In some embodiments, the pain is nociceptive pain. As used herein, “nociceptive pain” refers to pain caused by/arising from damage to body tissue(s). Nociceptive pain is distinguished from neuropathic pain, which is caused by damage to the nervous system. In some embodiments, the nociceptive pain arises from the soft tissues immediately along the iliac crest, posterior superior iliac spine, and/or sacrum. As used herein, “soft tissues” refer to supportive and/or connective body tissues, including muscles, tendons, ligaments, and aponeuroses. As used herein, “soft tissue attachments” refers to the attachment of soft tissue to bone and includes, without limitation, fleshy muscle attachments, fascial attachments, tendinous attachments, ligamentous attachments, and periosteum. In some embodiments, the source of pain in the soft tissues is an enthesopathy affecting the ligamentous, tendinous and aponeurotic attachments of the lower back and buttocks to the ilium, iliac crest, posterior superior iliac spine, and/or to the posterior and lateral sacral area. The source of pain in the soft tissues may also be of myofascial origin (originating from muscles or their fascial covering), or may be caused by periostitis (inflammation of the periosteum surrounding bones) of the iliac crest or sacrum. The source of pain may also arise from the fleshy attachments of the gluteal muscles, internal oblique, external oblique, transversus abdominus muscles, or the fascia lata attachment to iliac crest. In some embodiments the pain is not associated with a tendon or ligament.

In an aspect, the composition is administered by one or more injections. The composition may be administered by 1, 2, 3, 4, 5, 6, 7, 8, 9, 10, 15, 20, 25, or 30 injections, or by a number of injections within a range bounded by any of the foregoing. The injections may be repeated every 4-8 weeks indefinitely, or may be administered on an as needed basis indefinitely. In some embodiments, the composition is administered by 2-10 injections. The injections may be made using any suitable needle, including, without limitation, a 30 gauge, ½″ needle, and may deliver any suitable amount of HA. In some embodiments, each injection delivers about 0.25, 0.5, 0.75, 1, 1.5, 2, 2.5, 3, 4, 5, 6, 7, 8, 9, or 10 mL of the composition, or a volume of the composition within a range bounded by any of the foregoing. In some embodiments, each injection delivers about 0.5 mL to 1 mL of the composition. In some embodiments, each injection delivers 1, 2, 3, 4, 5, 6, 7, 8, 9, 10, 20, 30, 40, or 50 mg of HA, or an amount of HA within a range bounded by any of the foregoing. In some embodiments, each injection delivers 5 mg or more of HA. In some embodiments, each injection delivers 5 mg to 10 mg of HA. The amount of HA delivered may be independently selected for each injection.

In some embodiments, the injections are made into, or immediately adjacent to, the soft tissue attachments to the subject's iliac crest, posterior superior iliac spine, and/or sacrum. The injections may be made within about ½″ to about 1″ from the iliac crest, posterior superior iliac spine, and/or sacrum. Injections may acceptably be made into soft tissue immediately adjacent to the bony attachments to the palpated locations, as long as the injected volume of hyaluronic acid is sufficient to spread to these attachments. Larger injection volumes and dosages may allow for injection at greater distances from these locations. In some embodiments, the injections may include injection of the fleshy attachments of the gluteus minimis to the ilium.

In some embodiments, the composition is administered by two or more injections, spaced at an interval. The two or more injections may be spaced at an interval of about ¼″, ½″, ¾″, 1″, 1 ¼″, 1½″, 1¾″, 2″, 3″, or 4″, or by an interval within a range bounded by any of the foregoing. In some embodiments, the two or more injections are spaced at an interval of about ½″ to about 1.”

In some embodiments, the methods further comprise identifying one or more injection sites for the one or more injections, comprising palpating the ilium, iliac crest, posterior superior iliac spine, gluteus minimis origin, sacrum and/or coccyx. As used herein, “palpating” or “palpate” refers to a manual examination designed to assess by touch/feel one or more of the size, shape, firmness, location, consistency, texture, and/or tenderness of a body part. The palpation may be used to detect focal tenderness on or along the coccyx, sacrum, posterior superior iliac spine, iliac crest and/or lateral ilium of one or more of the following fleshy muscle attachments: the erector spinae, multifidus, gluteus maximus, gluteus medius, gluteus minimus, and/or external and internal oblique muscles. Palpation of these regions may identify pain originating from the thoracolumbar composite at its attachment sites to the sacrum and posterior superior iliac spine, and/or originating from the fascia lata at its attachment to the iliac crest, but can not distinguish between pain arising from fleshy muscle attachments or these complex connective tissue structures. However, MRI imaging can be used to identify pathology of the fascia lata attachment to the iliac crest, helping identify pain arising from the fascia lata in particular¹⁸. There are no reports of using MRI to identify pathology of the attachment of the thoracolumbar composite to the iliac crest, whereas ultrasound and MRI are routinely used to diagnose tendinopathy²¹. focal tenderness may be identified when the subject vocalizes pain, winces, or move their body in response to the pain elicited by firm palpation, and may reveal pain arising from fleshy muscle attachments, fascial attachments, tendinous attachments, ligamentous attachments, or periosteum.

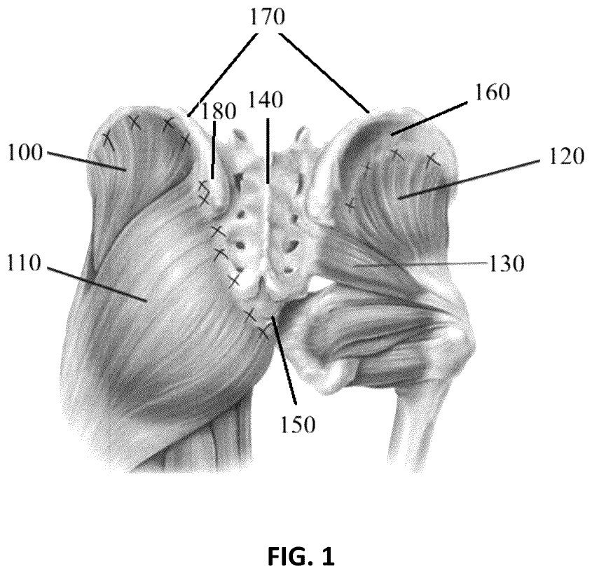

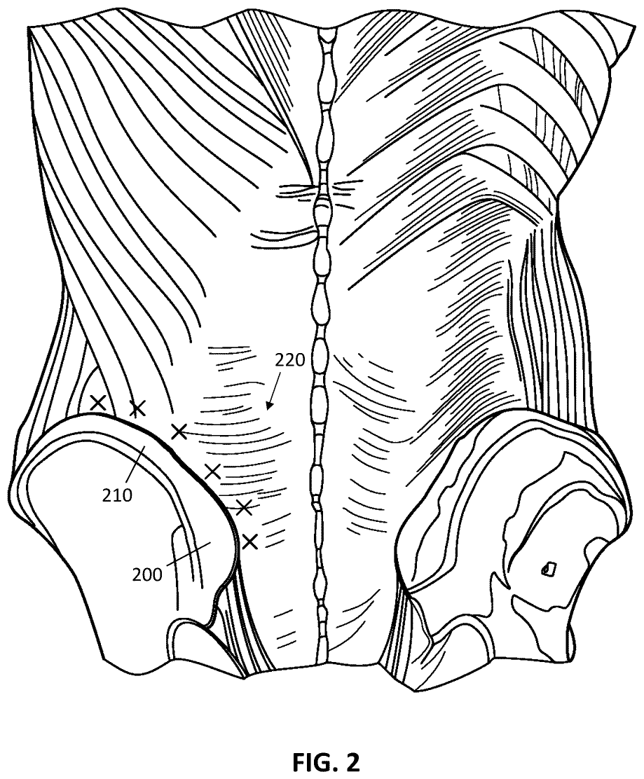

Any suitable palpation protocol may be used. For example, the palpation may be performed as follows: (1) beginning along the lower sacrum, the lateral edge of one side of the sacrum is palpated, from inferior (away from the head) to superior (toward the head), with palpation sites approximately ½″ to 1″ apart (see, FIG. 1 at [140]), (2) the area immediately lateral to the median crest of the same side of the sacrum is palpated from inferior to superior, with palpation sites approximately ½″ to 1″ apart, (3) the palpations described in (1)-(2) are repeated on the contralateral side of the sacrum; (4) the medial and lateral margins of the posterior superior iliac spine are palpated on each side from inferior to superior, with palpation sites approximately ½″ to 1″ apart (see, FIG. 1 at [180], FIG. 2 at [200]); (5) the superior and inferior margins of the posterior iliac crest on each side are palpated, proceeding up to the lateral aspect of the iliac crest or to the anterior superior iliac spine, with palpation sites approximately ½″ to 1″ apart (see, FIG. 1 at [170], FIGS. 2 at [210]), and (6) the lateral ilium on each side is palpated, with palpation sites approximately ½″ to 1″ apart (see, FIG. 1 at [160]). Exemplary sites for palpation on the left side or right side of a of a subject are illustrated in FIGS. 1-2 and are shown on each of the figures as marked with an “X”. Four sites for palpation and optional hyaluronic acid injection of the gluteus medius [100] are shown to the left, along the inferior margin of the iliac crest [170]. Five sites for palpation and optional hyaluronic acid injection of the gluteus maximus [110] are shown to the left side: two along the posterior superior iliac spine [180], three along the lateral edge/margin of the of the sacrum [140]. Four sites for palpation and optional hyaluronic acid injection of the gluteus minimus [120] are shown to the right side, along the ilium [160]. Six sites for palpation and optional hyaluronic acid injection of the interwoven thoracolumbar composite [220] are shown to the left: two along the medial and superior margins of the posterior superior iliac spine [200] and four along the superior margin of the iliac crest [210]. The order of palpating these sites may occur in any suitable order. For example, the beginning palpation at the anterior superior iliac spine and proceeding to the lower sacrum may help facilitate identification of the iliac crest in obese patients.

In some embodiments, areas of focal pain and/or tenderness identified by palpation are selected as injection sites, and the HA composition is injected into or immediately adjacent to the soft tissue attachments to these sites. The palpation may identify 1, 2, 3, 4, 5, 6, 7, 8, 9, 10, 15, 20, 25, or 30 injection sites, or a number of injection sites within a range bounded by any of the foregoing. In some embodiments, the one or more injections are made into 2-10 identified injection sites in a single treatment.

The composition may be administered using any suitable dosing scheme. For example, the composition may be administered daily, or once every 1, 2, 4, 6, 8, 10, 12, 16, 24, 36, or 52 weeks, or within a range bounded by any of the foregoing. In some embodiments, the composition is administered once per week. In other embodiments, the composition is administered once every 4 to 8 weeks, and the administration may be repeated every 4-8 weeks indefinitely, or may be administered on an as needed basis indefinitely. In some embodiments, the composition is administered once per week for 1-3 weeks, followed by a maintenance administration period comprising administration once every 8-16 weeks, 8-24 weeks, 8-52 weeks, or 16-52 weeks, or comprising administration on an as-needed basis. Prior to each administration, the palpation methods described herein may be repeated to identify sites for injection.

### Hip, Thigh, and Leg Pain

The present invention further provides methods for treating pain in a subject, wherein the subject is experiencing pain in the hip, thigh, and/or leg. The methods comprise administering the HA compositions described herein by one or more injections. In some embodiments, the pain is localized to the hip and/or thigh. In some embodiments, the patient is experiencing hip and/or thigh pain that radiates to the lower leg. In some embodiments, the hip, thigh, and/or leg pain is associated with lower back pain. In other embodiments, the hip, thigh, and/or leg pain is not associated with lower back pain.

The pain may be nociceptive pain. The nociceptive pain may arise from soft tissues immediately along the iliac crest, including the soft tissue attachments of the gluteus maximus, gluteus medius, external oblique, internal oblique, and/or transversus abdominus to the iliac crest. The pain may also arise from the attachments of the gluteus minimus to the ilium. In some embodiments, the subject has one or more co-morbid source of hip and/or thigh pain, such as osteoarthritis of the hip, labral tears of the hip joint, femoral neck fracture, femoro-acetabular impingement, avascular necrosis of the hip, iliopsoas tendonitis, proximal hamstring tendinopathy, greater trochanteric pain syndrome, meralgia paresthetica, and lumbar radiculopathy. A skilled artisan will readily appreciate the available methods for correctly diagnosing these other conditions. In other embodiments, the composition is administered to a subject who does not have additional co-morbid conditions contributing to the pain. These separate sources of hip, thigh or leg pain may be excluded by appropriate history, examination and diagnostic studies.

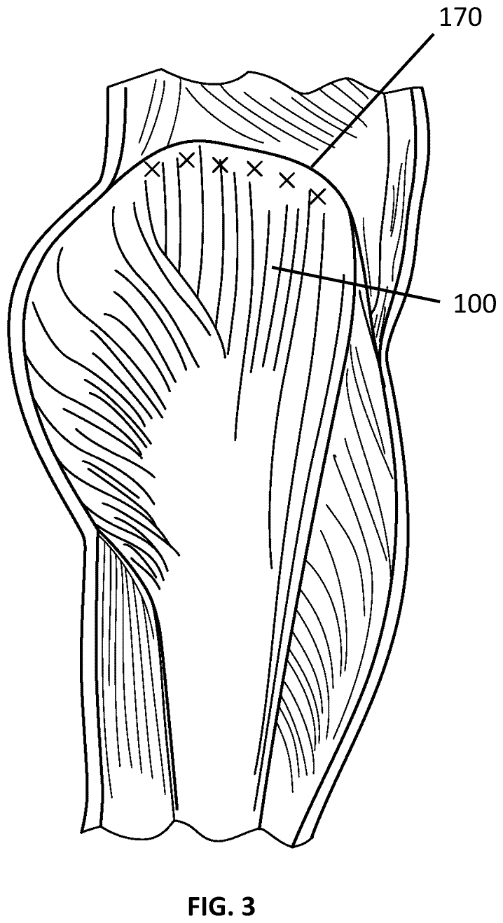

In some embodiments, the methods comprise identifying one or more injection sites for the one or more injections, comprising palpating the iliac crest and/or the origin of the gluteus minimus on the ilium. Any suitable palpation protocol may be used. For example, the palpation may be performed as follows: (1) the iliac crest on the same side of the hip or thigh pain is palpated along its inferior margin, from the anterior superior iliac spine to the border of the erector spinae on the posterior iliac crest, with palpated sites approximately 1″ apart and falling within the origin of the gluteus medius as it attaches to the iliac crest; and (2) the same region of the iliac crest is palpated along its superior margin, which palpated sites are approximately 1″ apart and reflect the origins of the external oblique, internal oblique, and transversus abdominus. Exemplary sites for palpation are illustrated in FIG. 3 and are shown as marked with an “X.”. Six sites for palpation and optional hyaluronic acid injection of the gluteus medius are shown along the iliac crest [170]. In some embodiments, areas of focal pain and/or tenderness identified by palpation are selected as injection sites, and the HA composition is injected into or immediately adjacent to the soft tissue attachments to these sites as described herein, using the provided dosing schemes.

### Coccygeal Pain

The invention further provides methods of treating pain in a subject, wherein the pain is experienced in the coccygeal region. The methods comprise administering the HA compositions described herein by one or more injections.

The pain may be nociceptive pain. The nociceptive pain may arise from soft tissues immediately along the coccyx, including the soft tissue attachments of the gluteus maximus. In some embodiments, the methods comprise identifying one or more injection sites for the one or more injections, comprising palpating the coccyx. Any suitable palpation protocol may be used. For example, the palpation may be performed as follows: (1) beginning along the lower coccyx and proceeding from inferior to superior, the coccyx is palpated, with palpated sites approximately 0.5″-1″ apart (see, FIG. 1 at [150]). Exemplary sites for palpation are illustrated in FIG. 1 and are shown as marked with an “X.” Two sites for palpation and optional hyaluronic acid injection of the gluteus maximus are shown to the left side. In some embodiments, areas of focal pain and/or tenderness identified by palpation are selected as injection sites, and the HA composition is injected into or immediately adjacent to the soft tissue attachments to these sites as described herein, using the provided dosing schemes.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument, and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### Exemplary Embodiments

Embodiment 1. A method of treating pain in a subject, the method comprising administering a composition comprising (a) a therapeutically effective amount of hyaluronic acid and (b) a pharmaceutically acceptable carrier, wherein the subject is experiencing pain in the hip, thigh, and/or leg, and wherein the composition is administered by one or more injections into, or immediately adjacent to, soft tissue attachments to the subject's ilium, iliac crest, posterior superior iliac spine, and/or sacrum.

Embodiment 2. The method of embodiment 1, wherein the pain is associated with lower back pain.

Embodiment 3. The method of embodiment 1, wherein the pain is not associated with lower back pain.

Embodiment 4. The method of any one of embodiments 1-3, wherein the pain is nociceptive pain.

Embodiment 5. The method of any one of embodiments 1-4, wherein the one or more injections are made into the soft tissue attachments within about ½″ to about 1″ from the ilium, iliac crest, posterior superior iliac spine, and/or sacrum.

Embodiment 6. The method of any one of embodiments 1-5, wherein the composition is administered by two or more injections along the subject's ilium, iliac crest, posterior superior iliac spine, and/or sacrum, and wherein the two or more injections are spaced at an interval of about ¼″ to about 4″.

Embodiment 7. The method of embodiment 6, wherein the two or more injections are spaced at an interval of about ½″ to about 1″.

Embodiment 8. The method of any one of embodiments 1-7, further comprising identifying one or more injection sites for the one or more injections, wherein the identifying comprises palpating the ilium, iliac crest, posterior superior iliac spine, and/or sacrum and selecting areas of pain or tenderness on palpating for injection.

Embodiment 9. The method of embodiment 8, wherein the one or more injections are made into 2-30 injection sites.

Embodiment 10. The method of embodiment 9, wherein the one or more injections are made into 2-10 injection sites.

Embodiment 11. The method of any one of embodiments 1-10, wherein the hyaluronic acid has a molecular weight of at least 500 kilodaltons (kDa).

Embodiment 12. The method of embodiment 11, wherein the hyaluronic acid has a molecular weight of 500 kDa to 730 kDa.

Embodiment 13. The method of any one of embodiments 1-12, wherein the hyaluronic acid is present in the composition at a concentration of 1 mg/mL to 30 mg/mL.

Embodiment 14. The method of embodiment 13, wherein the hyaluronic acid is present in the composition at a concentration of about 10 mg/mL.

Embodiment 15. The method of any one of embodiments 1-14, wherein each injection of the one or more injections delivers 5 mg or more of the hyaluronic acid.

Embodiment 16. The method of embodiment 15, wherein each injection of the one or more injections delivers 5 mg to 10 mg of the hyaluronic acid.

Embodiment 17. The method of any one of embodiments 1-16, wherein the composition is administered once per week.

Embodiment 18. The method of any one of embodiments 1-16, wherein the composition is administered once every four to twelve weeks.

Embodiment 19. The method of any one of embodiments 1-16, wherein the composition is administered once per week for 1 to 3 weeks, followed by once every 8 to 52 weeks.

Embodiment 20. The method of any one of embodiments 1-16, wherein the composition is administered once every 6 months to one year.

Embodiment 21. The method of any one of embodiments 1-20, wherein the one or more injections are made into one or more sites as shown in FIGS. 1-3 and/or are made into the soft attachments of the subject's the erector spinae, multifidus, gluteus maximus, gluteus medius, gluteus minimus, external oblique and/or internal oblique muscles.

Embodiment 22. A method of treating pain associated with a lower back surgery in a subject, comprising administering a composition comprising (a) a therapeutically effective amount of hyaluronic acid and (b) a pharmaceutically acceptable carrier, wherein the subject is experiencing pain in the lower back, buttocks, coccyx, sacral region, hip, thigh, and/or leg, wherein the pain has persisted following the lower back surgery, and wherein the composition is administered by one or more injections.

Embodiment 23. The method of embodiment 22, wherein the pain has worsened following the lower back surgery.

Embodiment 24. The method of embodiment 22 or 23, wherein the pain is nociceptive pain.

Embodiment 25. The method of any one of embodiments 22-24, wherein the one or more injections are made into, or immediately adjacent to, soft tissue attachments to the subject's ilium, iliac crest, posterior superior iliac spine, and/or sacrum.

Embodiment 26. The method of embodiment 25, wherein the one or more injections are made into the soft tissue attachments within about ½″ to about 1″ from the ilium, iliac crest, posterior superior iliac spine, and/or sacrum.

Embodiment 27. The method of any one of embodiments 22-26, wherein the composition is administered by two or more injections along the subject's ilium, iliac crest, posterior superior iliac spine, and/or sacrum, and wherein the two or more injections are spaced at an interval of about ¼″ to about 4″.

Embodiment 28. The method of embodiment 27, wherein the two or more injections are spaced at an interval of about ½″ to about 1″.

Embodiment 29. The method of any one of embodiments 22-28, further comprising identifying one or more injection sites for the one or more injections, wherein the identifying comprises palpating the ilium, iliac crest, posterior superior iliac spine, and/or sacrum and selecting areas of pain or tenderness on palpating for injection.

Embodiment The method of embodiment 29, wherein the one or more injections are made into 2-30 injection sites.

Embodiment 31. The method of embodiment 30, wherein the one or more injections are made into 2-10 injection sites.

Embodiment 32. The method of any one of embodiments 22-31, wherein the hyaluronic acid has a molecular weight of at least 500 kilodaltons (kDa).

Embodiment 33. The method of embodiment 32, wherein the hyaluronic acid has a molecular weight of 500 kDa to 730 kDa.

Embodiment 34. The method of any one of embodiments 22-33, wherein the hyaluronic acid is present in the composition at a concentration of 1 mg/mL to 30 mg/mL.

Embodiment 35. The method of embodiment 34, wherein the hyaluronic acid is present in the composition at a concentration of about 10 mg/mL.

Embodiment 36. The method of any one of embodiments 22-35, wherein each injection of the one or more injections delivers 1 mg or more of the hyaluronic acid.

Embodiment 37. The method of embodiment 36, wherein each injection of the one or more injections delivers 5 mg to 10 mg of the hyaluronic acid.

Embodiment 38. The method of any one of embodiments 22-37, wherein the composition is administered once per week.

Embodiment 39. The method of any one of embodiments 22-37, wherein the composition is administered once every 4 to 12 weeks.

Embodiment 40. The method of any one of embodiments 22-37, wherein the composition is administered once per week for 1 to 3 weeks, followed by once every 8 to 52 weeks.

Embodiment 41. The method of embodiment 40, once every 6 months to one year.

Embodiment 42. The method of any one of embodiments 22-41, wherein the one or more injections are made into one or more sites as shown in FIGS. 1-3 and/or are made into the soft attachments of the subject's the erector spinae, multifidus, gluteus maximus, gluteus medius, gluteus minimus, external oblique and/or internal oblique muscles.

Embodiment 43. A method of treating pain in a subject, the method comprising administering a composition comprising (a) a therapeutically effective amount of hyaluronic acid and (b) a pharmaceutically acceptable carrier, wherein the subject is experiencing coccygeal pain, and wherein the composition is administered by one or more injections into, or immediately adjacent to, soft tissue attachments to the subject's coccyx.

Embodiment 44. The method of embodiment 43, wherein the pain is nociceptive pain.

Embodiment 45. The method of embodiment 43 or 44, wherein the one or more injections are made into the soft tissue attachments within about ½″ to about 1″ from the coccyx.

Embodiment 46. The method of any one of embodiments 43-45, wherein the composition is administered by two or more injections along the subject's coccyx, and wherein the two or more injections are spaced at an interval of about ¼″ to about 4″ or about ½″ to about 1″.

Embodiment 47. The method of any one of embodiments 43-46, further comprising identifying one or more injection sites for the one or more injections, wherein the identifying comprises palpating the coccyx and selecting areas of pain or tenderness on palpating for injection.

Embodiment 48. The method of embodiment 47, wherein the one or more injections are made into 2-10 injection sites.

Embodiment 49. The method of any one of embodiments 43-48, wherein the hyaluronic acid has a molecular weight of at least 500 kilodaltons (kDa).

Embodiment 50. The method of embodiment 49, wherein the hyaluronic acid has a molecular weight of 500 kDa to 730 kDa.

Embodiment 51. The method of any one of embodiments 43-50, wherein the hyaluronic acid is present in the composition at a concentration of 1 mg/mL to 30 mg/mL.

Embodiment 52. The method of embodiment 51, wherein the hyaluronic acid is present in the composition at a concentration of about 10 mg/mL.

Embodiment 53. The method of any one of embodiments 43-52, wherein each injection of the one or more injections delivers 1 mg or more of the hyaluronic acid.

Embodiment 54. The method of embodiment 53, wherein each injection of the one or more injections delivers 5 mg to 10 mg of the hyaluronic acid.

Embodiment 55. The method of any one of embodiments 43-54, wherein the composition is administered once per week.

Embodiment 56. The method of any one of embodiments 43-54, wherein the composition is administered once every 4 to 12 weeks.

Embodiment 57. The method of any one of embodiments 43-54, wherein the composition is administered once per week for 1 to 3 weeks, followed by once every 8 to 52 weeks.

Embodiment 58. The method of any one of embodiments 43-54, once every 6 months to one year.

Embodiment 59. The method of any one of embodiments 43-58, wherein the one or more injections are made into one or more sites as shown in FIG. 1 and/or are made into the soft attachments of the subject's gluteus maximus to the coccyx.

### EXAMPLES

The Examples below demonstrate that HA injections into, or immediately adjacent to, the soft tissue attachments to the iliac crest, the posterior superior iliac spine, sacrum, and/or coccyx can be used to effectively treat patients with lower back, sacral, buttocks, hip, thigh, and/or leg pain. In the Examples, pain severity was assessed using a visual analog numerical rating scale ranging from 0 (no pain) to 10 (worst pain possible), and hyaluronic acid (HA) injections (Hyalgan™; 10 mg/mL, MW: 500,000 to 730,000 Daltons in 2 mL pre-filled syringes) were administered using a 30 gauge, ½″ needle.

### Example 1—Failed Lumbar Surgery Syndrome

In November 2023, a 49-year-old female patient reported lower back and right leg pain and weakness. A previous lumbar spine surgery with L3-S1 fusion conducted several years prior had helped the leg weakness she was experiencing prior to the surgery, but had persistent pain in the lower back on the right side, right thigh and hip and down into the right lower leg. The lower back pain severity was 6-7/10 (average) and 8-9 (maximum). Examination revealed sever tenderness along the attachments of the erector spinae along the right sacrum, right posterior superior iliac spine, as well along the posterior iliac crest and into the gluteus medius regions, and tenderness along her right greater trochanteric region. A recent CT of her lumbar spine revealed L3-S1 fusion with instrumentation, grade 1 anterolisthesis of L4 on L5 and L5 on S1, but no significant neuroforaminal or spinal canal stenosis. The patient had been treated with epidural steroid injections since the surgery, but the benefits lasted approximately a week.

On Feb. 5, 2024 hyaluronic acid (HA) injections for the lower back and right leg pains were administered as follows: 4 injections (0.5 mL each) along the right sacrum; 4 injections (1 mL each) along the right PSIS, with 2 medial injection sites and 2 lateral injection sites; 2 injections (1 mL each) along the right posterior iliac crest; 4 injections (0.5 mL each) along the right lateral iliac crest; and 6 (1 mL each) injections radially around the greater trochanter.

On Feb. 12, 2024, the patient reported that the back and leg pain were improved in the evening after the HA injections, and were quite significantly improved for 2 to 3 days, but returned to baseline distribution and severity within one week.

### Example 2—Hip, Thigh and Leg Pain

A 56-year-old female patient reported a greater than six-month history of right lower back pain, right hip and thigh pain, which radiates down to her right knee. An MRI of her lumbar spine revealed a disc protrusion at L1-2 but without significant associated foraminal or canal stenosis at that level. She reported previous treatment by pain management, including a transforaminal epidural injection, administered at the right L1-2 neural foraminal level without significant benefit. On exam, lower extremity strength, sensation and reflexes were intact, but there was distinct focal tenderness along the origin of her right gluteus medius, along the iliac crest on the right. There was only mild tenderness to the right greater trochanter. She was administered hyaluronic acid of a molecular weight of 500,000 to 750,000 Daltons, at a concentration of 10 mg/ml, in doses of approximately 1 mL spaced approximately 1″ apart from her anterior superior iliac spine to the superior portion of her posterior superior iliac spine. She reported significant improvement for two weeks and then the pain recurred.

### Example 3—Coccygeal Pain

A 57-year old woman was previously treated for refractory back and right hip/thigh pain with a dexamethasone injection to her right iliac crest. Symptoms had been improved for a couple of years, but had recurred over the preceding 3-4 weeks. However, this time she was having pain in her low back, right hip, thigh and tailbone region. She received an injection of dexamethasone to her right iliac crest and left and right lateral sacrum, and on follow up the back pain and right thigh pain improved, but she had persistent pain in her coccygeal region. She had palpable tenderness at the attachment site of gluteus maximus to her coccyx on each side, and received and injection of 10 mg of Hyalgan into the attachments of the left and right side of her coccyx. She additionally received injections of Hyalgan to the soft tissue attachments of her left and right PSIS for back pain. At follow up, both the back pain and coccygeal pain were significantly improved.

### Example 4—Coccygeal Pain

A 44-year-old male complained of having pain at the bottom of his tailbone, extending up towards his back a few inches, with significantly increased pain to sit, and had to carry around and sit on donut pads. This pain correlated with pain that extended into his perineal region for which he had seen urology and gastroenterology with an unremarkable evaluation, including an unremarkable CT abdomen and pelvis. He additionally complained of recurrent lower back pain that had previously been successfully treated with injections of hyaluronic acid to the soft tissues of his iliac crest. He received injection of hyaluronic acid Hyalgan brand with 0.5 cc doses given to the following locations:
Left lateral PSIS×1
Left medial PSIS×1
Right lateral PSIS×1
Right medial PSIS×1
Left lateral sacrum×4
Right lateral sacrum×4
Coccyx 0.5 cc on each side

Two weeks later, he stated the pain in his back and tailbone had significantly improved, averaging approximately 1 out of 10 in severity, with only occasional brief flare ups of the pain.

### REFERENCES

1. Hartvigsen et al., What low back pain is and why we need to pay attention, Lancet 2018 Jun. 9; 391 (10137): 2356-2367.
2. Karl H W et al., Superior and middle cluneal nerve entrapment: a cause of low back and radicular pain, Pain Physician 2022; 25: E503-521.
3. Morimote et al., Surgical treatment of superior cluneal nerve entrapment neuropathy, Journal of Neurosurgery Spine 2013; 19:71-75.
4. Hansson P. Neuropathic pain: clinical characteristics and diagnostic workup, European Journal of Pain, 2002; 6 Supplement A: 47-50.
5. Systemic Pharmacologic Therapies for Low Back Pain: A Systematic Review for an American College of Physicians Clinical Practice Guideline
6. Collee G. et al., Iliac crest pain syndrome in low back pain: frequency and features, Journal of Rheumatology 1991; 18 (7): 1064-7.
7. Njoo K. et al., Interobserver agreement on the iliac crest pain syndrome in general practice, Journal of Rheumatology 1995; 22 (8): 1532-5; Collee G. et al., Iliac crest pain syndrome in low back pain. A double blind, randomized study of local injection therapy, Journal of Rheumatology 1991; 18 (7): 1060-3.
8. Naim F. et al., Treatment of the chronic iliolumbar syndrome by infiltration of the iliolumbar ligament, Western Journal of Medicine 1982; 136 (4): 372-4.
9. Nayak B. et al., Recovering from nonspecific low back pain despair: Ultrasound-guided intervention in iliolumbar syndrome, The Indian journal of radiology & imaging 2020; 30 (4): 448-52).
10. Liu I. et al., Ultrasonographic diagnosis and guided treatment of erector spinae aponeurosis enthesopathy, Medical ultrasonography 2022; 24 (1): 120-21.
11. Wilkinson H, Injection therapy for enthesopathies causing axial spine pain and the “failed back syndrome”: a single blinded, randomized and cross-over study Pain Physician 2005; 8:167-
12. Neumann et al., High molecular weight hyaluronic acid inhibits advanced glycation endproduct-induced NF-kB activation and cytokine expression, FEBS Letters 1 999; 453:283-87.
13. Agostini F et al., Effects of hyaluronic acid injections on pain and functioning in patients affected by tendinopathies: A narrative review, Journal of Back and Musculoskeletal Rehabilitation, 2022; 35:949-961.
14. Nesporova K. et al., Injecting hyaluronan in the thoracolumbar fascia: A model study, International Journal of Biological Macromolecules, 2023; 253 (Pt 3): 126879.
15. Campa J A, Cross-Linked Hyaluronic Acid for the Management of Neuropathic Pelvic Pain, Practical Pain Management, 2018; 18 (5)
16. Amirdelfan K. et al., Allogeneic mesenchymal precursor cells treatment for chronic low back pain associated with degenerative disc disease: a prospective randomized, placebo-controlled 36-month study of safety and efficacy, The Spine Journal, 2021; 21 (2): 212-230
17. Benjamin M, Kumai T, Milz S, Boszczyk B M, Boszczyk A A, Ralphs J R. The skeletal attachment of tendons—tendon “entheses”. Comp Biochem Physiol A Mol Integr Physiol. 2002 December; 133 (4): 931-45
18. Deshmukh S, Abboud S F, Grant T, Omar I M. High-resolution ultrasound of the fascia lata iliac crest attachment: anatomy, pathology, and image-guided treatment. Skeletal Radiol. 2019 September;48 (9): 1315-1321
19. Fede C, Pirri C, Fan C, Petrelli L, Guidolin D, De Caro R, Stecco C. A Closer Look at the Cellular and Molecular Components of the Deep/Muscular Fasciae. Int J Mol Sci. 2021 Jan. 30; 22(3):1411
20. Mazzoleni M G, Maffulli N, Bardazzi T, Memminger M, Bertini F A, Migliorini F. Management of coccygodynia: talking points from a systematic review of recent clinical trials. Ann Jt. 2025 Jan. 21; 10:9
21. Weinreb J H, Sheth C, Apostolakos J, McCarthy M B, Barden B, Cote M P, Mazzocca A D. Tendon structure, disease, and imaging. Muscles Ligaments Tendons J. 2014 May 8; 4(1):66-73

What is claimed:

1. A method of treating pain in a subject, the method comprising administering a composition comprising (a) a therapeutically effective amount of hyaluronic acid and (b) a pharmaceutically acceptable carrier, wherein the subject is experiencing pain in the hip, thigh, and/or leg, and wherein the composition is administered by one or more injections into, or immediately adjacent to, soft tissue attachments to the subject's ilium, iliac crest, posterior superior iliac spine, and/or sacrum.

2. The method of claim 1, wherein the one or more injections are made into the soft tissue attachments within about ½″ to about 1″ from the ilium, iliac crest, posterior superior iliac spine, and/or sacrum.

3. The method of claim 1, wherein the composition is administered by two or more injections along the subject's ilium, iliac crest, posterior superior iliac spine, and/or sacrum, and wherein the two or more injections are spaced at an interval of about ¼″ to about 4″ or about ½″ to about 1″.

4. The method of claim 1, further comprising identifying one or more injection sites for the one or more injections, wherein the identifying comprises palpating the ilium, iliac crest, posterior superior iliac spine, and/or sacrum and selecting areas of pain or tenderness on palpating for injection.

5. The method of claim 4, wherein the one or more injections are made into 2-30 injection sites.

6. The method of claim 1, wherein:
(i) the hyaluronic acid has a molecular weight of at least 500 kilodaltons (kDa);
(ii) the hyaluronic acid is present in the composition at a concentration of 1 mg/mL to 30 mg/mL; and/or
(iii) each injection of the one or more injections delivers 1 mg or more of the hyaluronic acid.

7. The method of claim 6, wherein:
(i) the hyaluronic acid has a molecular weight of 500 kDa to 730 kDa;
(ii) the hyaluronic acid is present in the composition at a concentration of about 10 mg/mL; and/or
(iii) each injection of the one or more injections delivers 5 mg to 10 mg of the hyaluronic acid.

8. The method of claim 1, wherein the composition is administered:
(i) once per week;
(ii) once every 4 to 12 weeks;
(iii) once per week for 1 to 3 weeks, followed by once every 8 to 52 weeks; or
(iv) once every 6 months to one year.

9. The method of claim 1, wherein the one or more injections are made into one or more sites as shown in FIGS. 1-3.

10. The method of claim 1, wherein the one or more injections are made into the soft tissue attachments of the subject's erector spinae, multifidus, gluteus maximus, gluteus medius, gluteus minimus, external oblique and/or internal oblique muscles.

11. A method of treating pain associated with a lower back surgery in a subject, comprising administering a composition comprising (a) a therapeutically effective amount of hyaluronic acid and (b) a pharmaceutically acceptable carrier, wherein the subject is experiencing pain in the lower back, buttocks, coccyx, sacral region, hip, thigh, and/or leg, wherein the pain has persisted following the lower back surgery, and wherein the composition is administered by one or more injections.

12. The method of claim 11, wherein the one or more injections are made into, or immediately adjacent to, soft tissue attachments to the subject's ilium, iliac crest, posterior superior iliac spine, and/or sacrum.

13. The method of claim 12, wherein the one or more injections are made into the soft tissue attachments within about ½″ to about 1″ from the ilium, iliac crest, posterior superior iliac spine, and/or sacrum.

14. The method of claim 13, wherein the composition is administered by two or more injections along the subject's iliac crest, posterior superior iliac spine, and/or sacrum, and wherein the two or more injections are spaced at an interval of about ¼″ to about 4″ or about ½″ to about 1″.

15. The method of claim 12, further comprising identifying one or more injection sites for the one or more injections, wherein the identifying comprises palpating the ilium, iliac crest, posterior superior iliac spine, and/or sacrum and selecting areas of pain or tenderness on palpating for injection.

16. The method of claim 15, wherein the one or more injections:
(i) are made into 2-30 injection sites;
(ii) are made into one or more sites as shown in FIGS. 1-3; and/or
(iii) are made into the soft tissue attachments of the subject's erector spinae, multifidus, gluteus maximus, gluteus medius, gluteus minimus, external oblique and/or internal oblique muscles.

17. The method of claim 11, wherein:
(i) the hyaluronic acid has a molecular weight of at least 500 kilodaltons (kDa);
(ii) the hyaluronic acid is present in the composition at a concentration of 1 mg/mL to 30 mg/mL; and/or
(iii) each injection of the one or more injections delivers 1 mg or more of the hyaluronic acid.

18. A method of treating pain in a subject, the method comprising administering a composition comprising (a) a therapeutically effective amount of hyaluronic acid and (b) a pharmaceutically acceptable carrier, wherein the subject is experiencing coccygeal pain, and wherein the composition is administered by one or more injections into, or immediately adjacent to, soft tissue attachments to the subject's coccyx.

19. The method of claim 18, wherein the one or more injections are made into the soft tissue attachments within about ½″ to about 1″ from the coccyx.

20. The method of claim 18, wherein the composition is administered by two or more injections along the subject's coccyx, and wherein the two or more injections are spaced at an interval of about ¼″ to about 4″ or about ½″ to about 1″.

21. The method of claim 20, further comprising identifying one or more injection sites for the one or more injections, wherein the identifying comprises palpating coccyx and selecting areas of pain or tenderness on palpating for injection.

22. The method of claim 21, wherein the one or more injections
(i) are made into 2-10 injection sites;
(ii) are made into one or more sites as shown in FIG. 1; and/or
(iii) are made into the soft tissue attachments of the subject's gluteus maximus to the coccyx.

23. The method of claim 18, wherein:
(i) the hyaluronic acid has a molecular weight of at least 500 kilodaltons (kDa);
(ii) the hyaluronic acid is present in the composition at a concentration of 1 mg/mL to 30 mg/mL; and/or
(iii) each injection of the one or more injections delivers 1 mg or more of the hyaluronic acid.
